(19) United States
(12) Patent Application Publication
(10) Pub. No.: US 2005/0037518 A1
(43) Pub. Date: Feb. 17, 2005

(54) Anti-inflammatory signal molecules and exercise

(76) Inventor: George B. Stefano, Melville, NY (US)

Correspondence Address: HOFFMAN WARNICK & D'ALESSANDRO, LLC, 3 E-COMM SQUARE, ALBANY, NY, 12207, US

(21) Appl. No.: 10/916,655

(22) Filed: Aug. 12, 2004

Related U.S. Application Data
(60) Provisional application No. 60/494,505, filed on Aug. 12, 2003.

(51) Int. Cl. G01N 33/543

(52) U.S. Cl. 436/518

(57) ABSTRACT

The present invention provides methods for assessing the effect of exercise on an individual by measuring changes in the concentration of a compound in the individual's blood. Specifically, the invention measures changes in the concentration of an anti-inflammatory signal molecule or endogenous morphine. Anti-inflammatory signal molecules include, for example, adrenocorticotropin (ACTH), cortisol, and interleukin-10 (IL-10). A first embodiment of the invention provides a method for assessing the effect of exercise on an individual comprising the steps of measuring a concentration of at least one compound in the individual's blood before exercise, measuring a concentration of the at least one compound in the individual's blood after exercise, and comparing the concentrations of the at least one compound.

### CROSS REFERENCE TO RELATED APPLICATIONS

The current application claims the benefit of co-pending U.S. Provisional Application No. 60/494,505 filed Aug. 12, 2003, which is hereby incorporated herein by reference.

### BACKGROUND OF THE INVENTION

1. Technical Field

The present invention relates generally to anti-inflammatory signal molecules and more particularly to the use of anti-inflammatory signal molecules to measure the effect of an exercise regimen on an individual. Anti-inflammatory signal molecules include anti-inflammatory cytokines and signal molecules, such as adrenocorticotropin (ACTH), cortisol, and Interleukin-10 (IL-10). The invention may be employed to treat healthy individuals and to treat patients exhibiting immune system alterations due to illness or disease.

2. Related Art

Idiopathic Parkinson's Disease (PD) is a degenerative disorder of the human central nervous system. Several pathogenic mechanisms have been proposed to account for the degeneration of dopaminergic neurons: metabolic factors, oxidative stress and mitochondrial dysfunction. The main anatomic features of brains from PD patients include a diminished number of melanized dopaminergic cells in the substantia nigra (SN) and in related brain stem nuclei, a decrease in the dopamine content in nigrostriatal and mesolimbic pathways, the presence of Lewy bodies, and the deposition of neuromelanin.

The perturbation of several neurotransmitters and neuropeptides has been reported in PD, indicating a more complicated and widespread pathology. It has been known for many years that immune system alterations occur in patients with Parkinson's disease (PD) and the role of immune system mechanisms is an important area of investigation. Death or injury to neurons leads to the presence of many pro-inflammatory cytokines. Increases in lymphocyte populations in cerebrospinal fluid and blood, immunoglobulin synthesis, and cytokine and acute phase protein production have been observed in patients with PD. This process resembles a classic inflammatory process, except that the participation of macrophages and lymphocytes is lacking or minimal.

Thus, a need exists for methods of treating diseases or conditions characterized by inflammatory responses by increasing the production and/or concentration of anti-inflammatory molecules.

### SUMMARY OF THE INVENTION

The present invention provides methods for assessing the effect of exercise on an individual by measuring changes in the concentration of a compound in the individual's blood. Specifically, the invention measures changes in the concentration of an anti-inflammatory signal molecule or endogenous morphine. Anti-inflammatory signal molecules include, for example, adrenocorticotropin (ACTH), cortisol, and interleukin-10 (IL-10).

A first embodiment of the invention provides a method for assessing the effect of exercise on an individual comprising the steps of measuring a concentration of at least one compound in the individual's blood before exercise, measuring a concentration of the at least one compound in the individual's blood after exercise, and comparing the concentrations of the at least one compound.

A second embodiment of the invention provides a method for treating at least one of a disease and a condition in an individual comprising the steps of measuring a concentration of at least one compound in the individual's blood before exercise, directing the individual in an exercise, measuring a concentration of the at least one compound in the individual's blood after exercise, comparing the concentrations of the at least one compound, and modifying the exercise to increase the concentration of the at least one compound.

A third embodiment of the invention provides a method of increasing a concentration of at least one compound in an individual's blood including an exercise regimen comprising the steps of measuring the individual's resting heart rate, directing the individual in a first aerobic exercise until the individual's heart rate stabilizes, directing the individual to rest until the resting heart rate is achieved, directing the individual in a second aerobic exercise until the individual's heart rate is about 10 beats per minute greater than the stabilized heart rate achieved during the first aerobic exercise, directing the individual to rest until the resting heart rate is achieved, directing the individual in a third aerobic exercise such that the heart rate achieved in the second aerobic exercise is achieved in less time, directing the individual to rest until the resting heart rate is achieved, directing the individual in a fourth aerobic exercise until the individual's heart rate is about 10 beats per minute greater than the heart rate achieved during the second aerobic exercise, directing the individual to rest until the resting heart rate is achieved, and directing the individual in a fifth aerobic exercise until either the individual's heart rate has plateaued or the exercise has continued for one minute.

The foregoing and other features of the invention will be apparent from the following more particular description of embodiments of the invention.

### BRIEF DESCRIPTION OF THE DRAWINGS

The embodiments of this invention will be described in detail, with reference to the following figures, wherein like designations denote like elements, and wherein:

FIG. 1 shows a histogram of ACTH and cortisol plasma levels for four groups of patients.

FIG. 2 shows a histogram of IL-10 and IL-6 plasma levels for four groups of patients.

FIG. 3 shows a histogram of morphine plasma levels for four groups of patients.

FIG. 4 shows chromatograms of morphine in the plasma of Parkinson's Disease patients and a morphine standard.

FIG. 5 shows mass spectrometric graphs of an external morphine standard, an HPLC fraction of plasma containing morphine, fragmentation of a morphine molecule, and fragmentation of morphine from a patient's plasma.

FIG. 6 shows a flow diagram of an exercise regimen according to the invention.

### DETAILED DESCRIPTION OF THE INVENTION

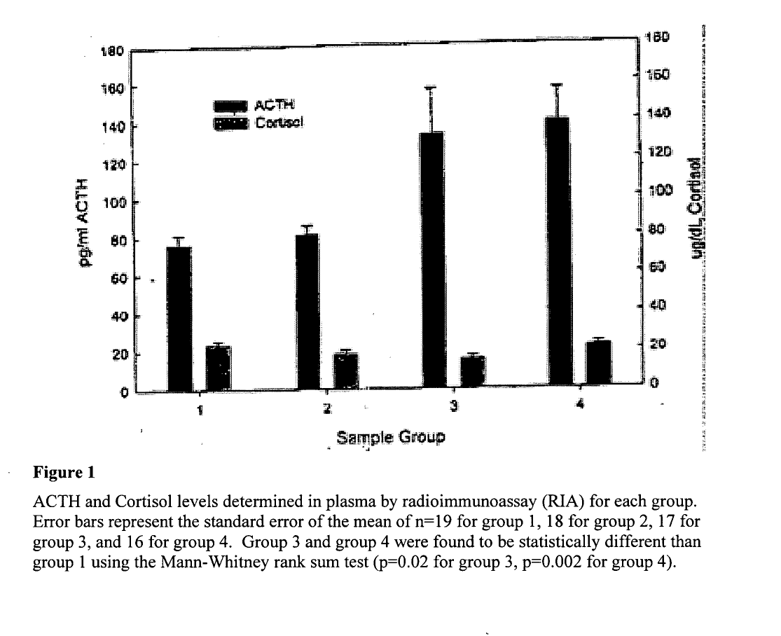

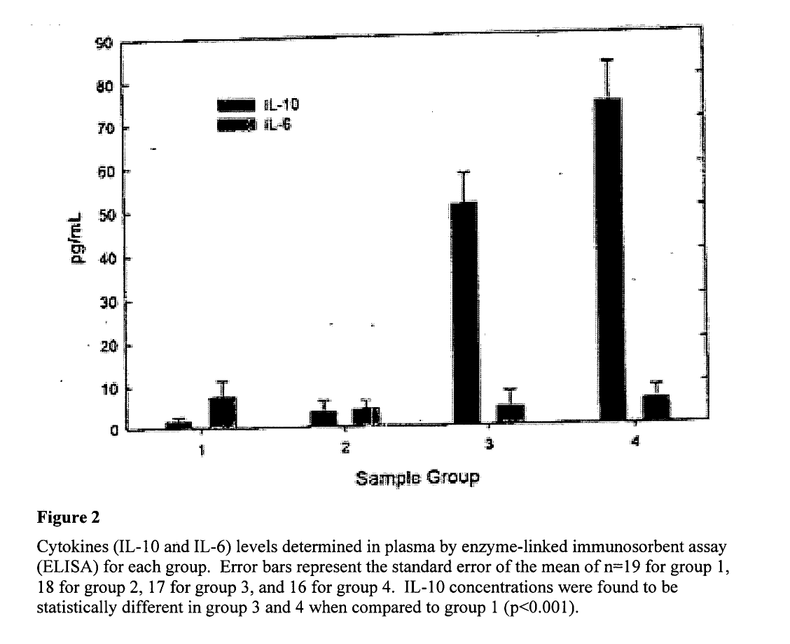

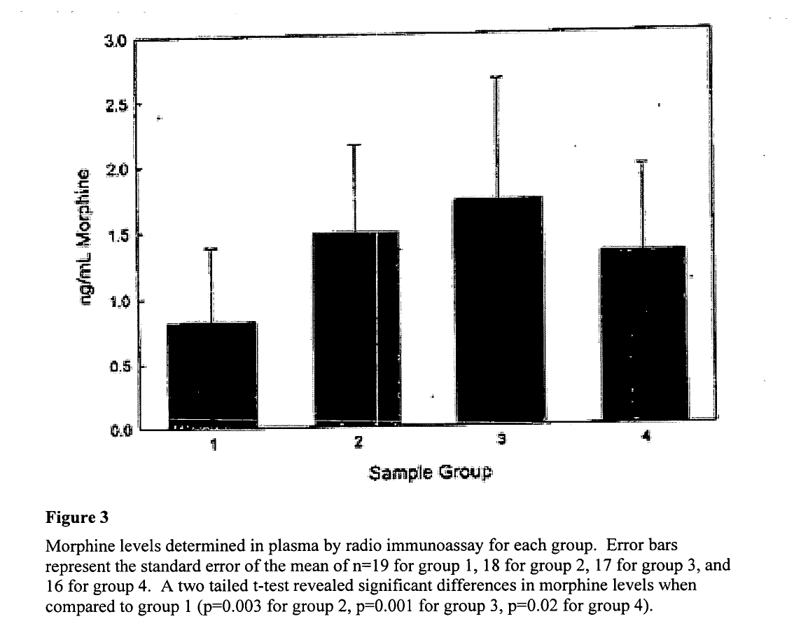

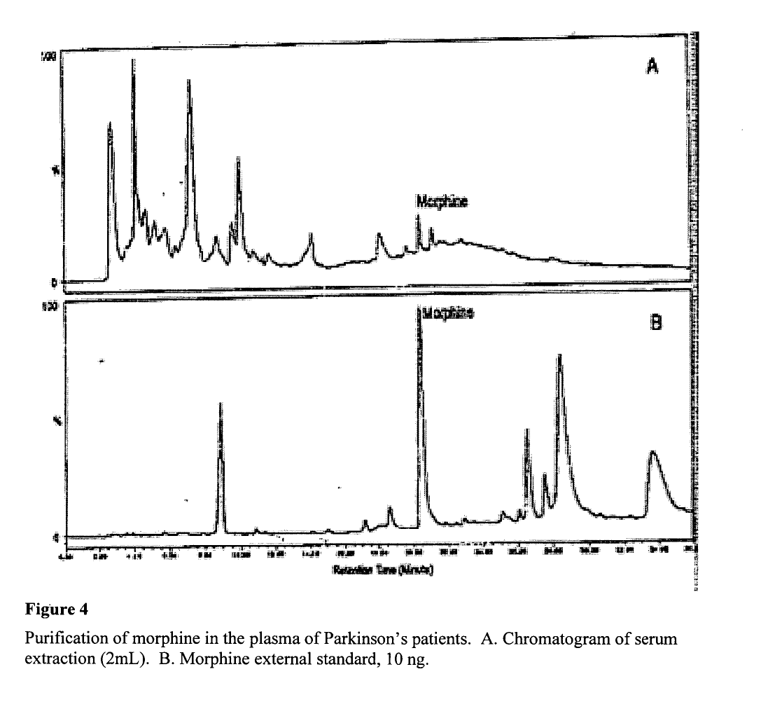

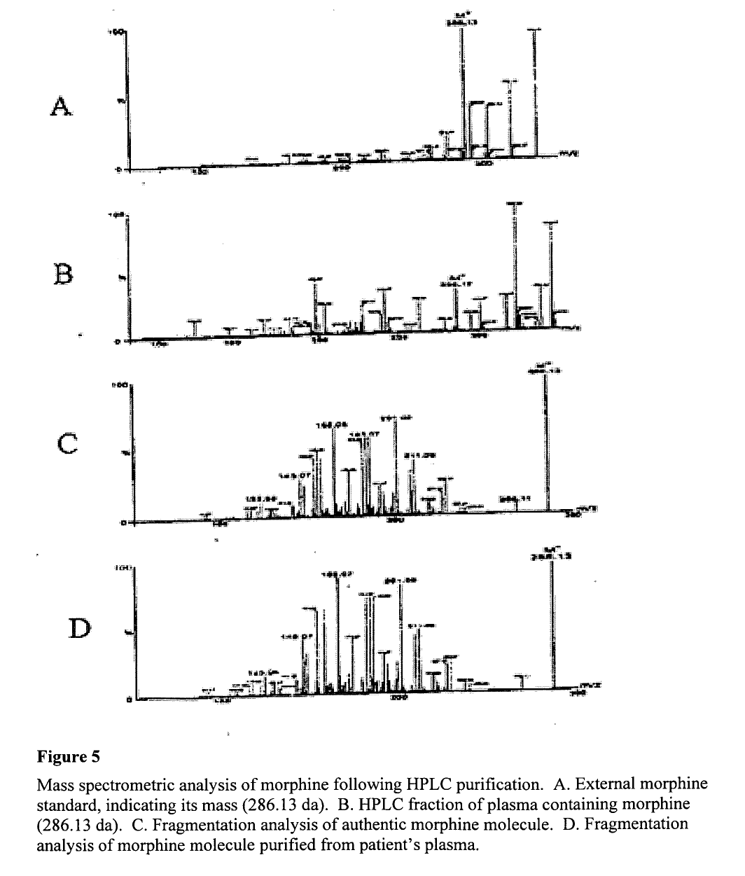

In studying the effects of exercise protocols on patients with PD, it has been found that mild exercise over a period of months resulted in a significant increase in plasma anti-inflammatory signal molecules, see FIGS. 1-5, and a concomitant enhancement in motor skills and mood elevation. Consequently, by increasing the formation of anti-inflammatory signal molecules in the blood, some of the clinical characteristics of PD may be alleviated, including those associated with loss of motor function and mood. Likewise, increasing the formation of such molecules in the blood will have a positive effect on the treatment of illnesses and diseases which are characterized by increases in cerebral spinal fluid and blood lymphocyte populations, immunoglobulin synthesis, and cytokine and acute phase protein production. It follows therefore that increasing the formation of anti-inflammatory signal molecules in the blood of healthy individuals will likewise positively affect the individual's motor function, mood and health maintenance by increasing the populations of anti-inflammatory molecules in the blood.

Such anti-inflammatory signal molecules include, without limitation, ACTH, IL-10 and endogenous morphine. Three general classes of cell surface opioid receptors (kappa, delta and mu (μ)) have been described. Receptors exhibiting high binding specificity for morphine have been designated μ opioid receptors. Detailed analysis has revealed the existence of multiple μ opioid receptor subtypes. Isolated nucleic acid sequences encoding various μ receptors and polypeptides comprising μ receptors (and referred to here as “μ3 opioid receptor(s)”) are disclosed in detail in PCT Patent Publication WO 99/24471, published 20 May 1999.

Thus, in one aspect, the invention provides a method of monitoring the effects of an exercise protocol on an individual. The method comprises: (a) measuring μ3 opiate receptor expression in an individual prior to the start of an exercise protocol, (b) measuring μ3 opiate receptor expression in the individual after the start of the protocol, and (c) comparing the level of μ3 opiate receptor expression in the samples.

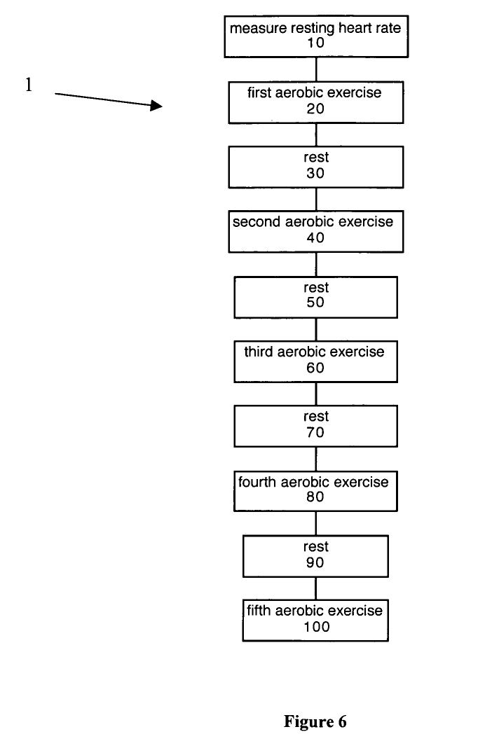

The exercise protocol may be individually tailored for each patient by the determination of initial baseline heart rate (HR) measurements. Referring to FIG. 6, baseline heart rates measurements can be established by having the patient perform a series of five cycles of aerobic exercise using, for example, an exercise bicycle, a trampoline, or a stair climbing apparatus, with rest cycles between each exercise cycle that allow HR to return to its resting HR. First, resting heart rate is measured 10. Then first aerobic exercise 20 is initiated and consists of mild aerobic exercise (up to 3 on the 10 point Borg scale) until HR stabilization. Upon stabilization, rest and recovery 30 are initiated until HR returns to resting rate. Second aerobic exercise 40 is then begun by exercising to increase HR 10 beats per minute (bpm) over that attained in first aerobic exercise 20. Once attained, rest and recovery 50 is again initiated. Third aerobic exercise 60 consists of exertion to reach the target HR of second aerobic exercise 40 at an increased rate. Fourth aerobic exercise 80 consists of exercise to increase HP 10 bpm over that attained in second aerobic exercise 40. Rest and recovery 90 is again initiated. Fifth aerobic exercise 100 consist of vigorous exertion to at least 9 on the 10 Borg scale and results in a maximum HR measurement (in bpm).

Once the baseline determinations are made, subjects begin a prescribed course of cyclic exercise three times per week monitored by a neurologist, a nurse practitioner, and a trainer. The cycles are designed to increase the capacity for cardiovascular acceleration and recovery, adjusted according to base heart-rate range and performance, in the context of normal circadian rhythm. This design produces a progressive set of cycles, where heart-rate targets increase (or rate of speed to targets increases) for each subsequent cycle within a set and the peak heart rate targets increase as the cycles move from morning to afternoon. The peak heart rate achieved by each patient during baseline fifth aerobic exercise 100 is used as the initial maximum heart rate. Subsequent target heart rates are set as increasing percentages of this initial maximum.

The initial cycle in the first set of training cycles uses a target heart rate of 60-80%, preferably 70%, of the patient's peak rate and the final cycle of the first set of training cycles used a target heart rate 85-90%, preferably 85%, of the patient's peak rate. The cycles are typically performed between 0600 and 0900 hrs (6-9 a.m.), three days per week for twelve weeks. The procedure is identical to that used for the baseline determinations, except that the target heart-rates are pre-determined and the number of session are varied. A trainer tracks the patients as they move through the exercise-recovery cycles.

The number of cycles per session may decreases over each week, but both the initial and peak heart-rate targets should increase (the initial cycle target should increase 3-4 beats and the peak target rate should increase 2-3 beats per session) and continue over the course of the twelve weeks, effectively increasing exertion. The intervening cycles in each session should be designed to move the heart-rate to intermediary levels, with varying levels of exertion. The targets serve as guides; the exertion may be terminated when the target heart-rate is either reached or exceeded, or the time of exertion exceeds one minute. During the second week, the peak target heart-rate is 90% of the initial maximum, the peak heart-rate increasing 2 beats each succeeding session. The third week, the peak heart-rate is 95% of the initial maximum. In addition, the exertion rate may be increased to 9 on the 10-point Borg Scale at times during the third and following sessions. During the fourth week, the first session may include two exertion rate increases (“spikes”) and the following two sessions may consist of all spikes, after the initial warm up. If this results in the heart-rate going beyond the baseline maximum, a new baseline maximum is established and used in the subsequent weeks of testing. The subsequent weeks of testing employ the following weekly protocol: The first session of the week comprises seven cycles, the second comprises 6 cycles and the third comprises 5 cycles. This is repeated the following week. The third week, each of the three session comprise 5 cycles. The fourth week, the first session comprises 6 cycles and the second and third sessions comprise 5 cycles each. The subsequent two months repeat the pattern of the first month.

The exercise method may be employed to treat illnesses or diseases in humans that are characterized by inflammatory processes, sepsis conditions viral infections and cardiovascular diseases. Exemplary are arthritis, pericarditis, vasculitis, lupus, bronchitis, phrenitis and HIV infections, Parkinson's disease, schizophrenia, Alzheimer's disease, Chrone's disease, colitis, diabetes mellitus, autoimmune diseases, toxoplasmosis, asthma, and multiple sclerosis. Employment of the exercise method of the invention increases the release of nitric oxide, which promotes responses that are beneficial to the individual or patient. For example, nitric oxide release can promote anti-inflammatory and immunosuppresive responses, prevent septic shock, promote anti-viral activity and reduce or prevent atherosclerosis. The exercise method of the invention stimulates μ3 receptor activity, thereby increasing the release of nitric oxide.

Thus, in another aspect, the invention provides a method for treating an anti-inflammatory disease or condition in a human patient by (a) measuring μ opiate receptor expression in a blood sample from the patient prior to the start of an exercise protocol, (b) directing the patient in an exercise protocol, (c) measuring μ opiate receptor expression in a blood sample from the patient at least once after the start of the exercise protocol and within one week prior to the termination of the exercise protocol, (c) comparing the level of μ opiate receptor expression in the samples, and optionally, (d) modifying the exercise protocol to increase the amount of anti-inflammatory molecules in the blood. μ opiate receptor expression should be measured just prior to the start of the exercise protocol in order to generate a baseline measure. After the initial measurement, subsequent measurements should be generated to track progress and enable modification of the exercise protocol to elicit desirable levels of circulating blood anti-inflammatory molecules. The subsequent measurements can be made at any time during the course of the exercise protocol, but in no case should any subsequent measurements be taken more than one week after the termination of the exercise protocol. Preferably, once monthly subsequent measurements should be taken, most desirably at the end of each month's exercise regimen. The final measurement should most desirably be taken immediately after the end of the exercise protocol.

The method may likewise be employed to induce the production of anti-inflammatory molecules in human blood. In this aspect, the invention provides a method for increasing levels of anti-inflammatory molecules in the blood by (a) measuring mu opiate receptor expression in a blood sample from an individual prior to the start of an exercise protocol, (b) measuring mu opiate receptor expression in a blood sample from the individual at least once after the start of the exercise protocol and within one week after temination of the exercise protocol, (c) comparing the level of mu opiate receptor expression in the samples and (d) modifying the exercise protocol to increase the amount of anti-inflammatory molecules in the blood.

Generally, the exercise protocol should result in increasing levels of anti-inflammatory molecules from their baseline levels to 1 to 2 ng/mL for morphine, 0 to 14.1 pg/mL for IL-10 serum, and 1.3 to 15.6 pg/mL for IL-10 plasma.

The following Examples show that anti-inflammatory molecules significantly increase in the plasma of PD patients' months after initiating and sustaining a mild exercise protocol. The level of ACTH and IL-10 in the blood of these patients increased significantly after one month on the exercise protocol. An opiate-like compound isolated and extracted from patient plasma was identified as morphine and endogenous levels of it also significantly increased after the exercise protocol. Pro-inflammatory cytokine plasma levels of IL-6 did not change and levels of IL-1 and TNF-α were not detected during the entire examination period.

Taken together, these results indicate that mild exercise programs induce the formation of signaling molecules associated with immune, vascular and neural down regulation. This finding supports the theory that an underlying chronic immune, vascular and/or neural process can be alleviated by the induction of these anti-inflammatory signal molecules via the employment of a mild exercise regimen. Increased production of these anti-inflammatory signal molecules should likewise be induced in healthy patients via employment of a mild exercise regimen.

Induction of these anti-inflammatory signal molecules stimulated by mild exercise is monitored by an assay that measures a biological response induced by a μ3 opiate receptor. Nucleic acid sequences encoding mu3 opiate receptors, polypeptides constituting μ3 opiate receptors, assays employing such receptors and assays identifying agonists and antagonists of such receptors are known in the art and described in detail in PCT Patent Publication WO 99/24471, published 20 May 1999, incorporated herein by reference.

### EXAMPLE 1

### Exercise Regimen

Fourteen male and five female patients previously diagnosed and undergoing treatment for moderate to severe PD were subjected to a cyclic exercise protocol designed to generate a series of parabolic-like waves of cardiovascular exercise and recovery. The effect of this protocol in healthy subjects is reported elsewhere. Cycles were tailored to the individual subjects following an initial, baseline determination of cardiovascular responsiveness during short burst of exercise followed by recovery. During baseline testing, patients were evaluated with the Uniform Parkinson Disease Rating Scale (UPDRS). Five second averaged heart rates were monitored and recorded continuously using a Polar NV heart-rate monitor watch and chest strap (Polar Electro Inc., NY).

Once the baseline determinations were made, subjects began a prescribed course of cyclic exercise three times per week monitored by a neurologist, a nurse practitioner, and a trainer. The cycles were designed to increase the capacity for cardiovascular acceleration and recovery, adjusted according to base heart-rate range and performance, in the context of normal circadian rhythm. This design produces a progressive set of cycles, where heart-rate targets increase (or rate of speed to targets increases) for each subsequent cycle within a set and the peak heart rate targets increase as the cycles move from morning to afternoon, as described below.

The exercise protocol included two stages: (1) a baseline determination stage and (2) the actual training cycle protocol. In the baseline determination stage, patients refrained from caffeine and meals for 3 hours prior to exercise. Before beginning, the patients were briefed on the 5-cycle baseline protocol, familiarized with the exercise equipment, and instructed on the relaxation response. Then the patients sat quietly for a seven-minute period. After three minutes, patients were instructed to initiate the relaxation response. After two more minutes, the blood pressure (right arm) of each patient was measured. At the end of the seven minute period, patients were instructed to take a deep breath and begin the first cycle of the baseline determination.

The first cycle of the baseline determination consisted of riding an exercise bicycle (a Schwinn Airodyne), walking on stairs, or bouncing on a trampoline (Body by Jake), depending on patient preference and physical capability under close supervision and at an easy pace of 3 on the 10-point Borg scale until heart-rate stabilization (less than one minute). Heart-rate stabilization was determined by tracking beats per minutes with a second Polar NV watch. When heart-rate had stabilized, the patient ceased the activity, sat, and began the relaxation response.

When a patient's heart-rate had returned to its resting heart-rate, a second exercise and relaxation cycle was initiated. This cycle was identical to the initial cycle, except that exertion by the patient was increased sufficient to increase patient heart-rate by 10 beats per minute over that attained in the initial cycle. Exertion was stopped and the recovery phase initiated when the patient reached the target of 10 beats per minute over baseline or after one minute. After heart-rate stabilization at baseline levels during the relaxation phase, a third cycle commenced with the same target heart-rate as in cycle two but with an increased pace of exertion to reach the target more swiftly, followed by a recovery phase to return heart-rate to baseline levels. Cycle 4 increased the target heart-rate 10 beats per minute over cycle 2. Cycle 5 consisted of very vigorous exertion (9, on the 10-point Borg Scale), done until the heart-rate plateaued or for one minute, followed by a final recovery phase. Blood pressure readings were taken five and ten minutes after peak heart-rate was attained. The patient then resumed normal activity.

The foregoing baseline data was used to determine the training cycle protocol target heart-rates for each of the 19 patients in the study. The peak heart-rate achieved by each patient during baseline cycle 5 was used as the initial maximum heart-rate. Subsequent target heart-rates were set as increasing percentages of this initial maximum.

The initial cycle in the first set of training cycles used a target heart-rate of 70% of the patient's peak rate and the final cycle of the first set of training cycles used a target heart-rate of 85% of the patient's peak rate. The cycles were performed between 0600 and 0900 hrs (6-9 a.m.), except where indicated in Table 1, three days per week for twelve weeks. The procedure was identical to that used for the baseline determinations, except that the target heart-rates were pre-determined and the number of session varied. A trainer tracked the patients as they moved through the exercise-recovery cycles.

The number of cycles per session decreased over each week, but both the initial and peak heart-rate targets increased (the initial cycle target increased 3-4 beats and the peak target rate increased 2-3 beats per session) and continued over the course of the month, effectively increasing exertion. See Table 1. The intervening cycles in each session were designed to move the heart-rate to intermediary levels, with varying levels of exertion. The targets served as guides; the exertion was terminated when the target heart-rate was either reached or exceeded, or the time of exertion exceeded one-minute. During the second week, the peak target heart-rate was 90% of the initial maximum, the peak heart-rate increasing 2 beats each succeeding session. The third week, the peak heart-rate was 95% of the initial maximum. In addition to the exertion rate was increased to 9 on the 10-point Borg Scale at times during the third and following sessions. During the fourth week, the first session included two exertion rate increases (“spikes”) and the following two sessions consisted of all spikes, after the initial warm up. If this resulted in the heart-rate going beyond the baseline maximum, a new baseline maximum was established and used in the two subsequent month(s) of testing. Months 2 and 3 of testing employed the same weekly protocol as summarized in Table 1, below.

TABLE 1
Monthly Overview of Cycle Protocol

| | Mon. Cycles/Set | Wed. Cycles/Set | Fri. Cycles/Set |
|---|---|---|---|
| Week 1 | 7 | 6 | 5 |
| Week 2 | 7 | 6 | 5 |
| Week 3 | 5 | 5 | 5 |
| (0900-1200 hrs) | | | |
| Week 4 | 6 | 5 | 5 |
| (1500-1800 hrs) | | | |

### EXAMPLE 2

### Performance Analysis

A. Heart-Rate Range

Heart-rate range is relatively constricted in elderly patients. They have a narrow resting range and a peak exertion range. Heart-rate range (HR) targets were designed to improve that range, moving the resting HR lower and the peak HR higher. In healthy subjects, the training cycle of Example 1 produced significant gains in heart-rate range and in heart-rate variability in healthy subjects (Goldsmith, et al 2001, submitted, data not shown). Overall, there was a small increase in heart-rate range, >5%. In some subjects, HR range expanded dramatically, >200%.

B. Disease State Improvement

Parkinson's disease state improved in 5 participants, as indicated by Hoehn and Yahr scale (H & Y scale) (n=14).

C. UPDRS Motor Function

Significant improvement in UPDRS Motor Function was seen in a number of the participants (n=15).

D. Blood Pressure Results

Diastolic Pressure measurements of the participants in the study were taken both before each exercise session and after each exercise session. Diastolic Pressure taken before exercise showed a mean decrease of 8.8% from the beginning to the end of the experiment. Diastolic Pressure taken after exercise showed a mean decrease of 17% from the beginning to the end of the experiment. Both decreases were significant. Systolic Pressures did not show a significant change following cycles training.

E. Immunological Data

Immunological function was assessed over a number of parameters. Blood draws were taken every four weeks during the study and 8 weeks post-study. Data (being reported separately) suggest that cycles improve immune function and that pro-inflammatory responses are not activated by this form of exertion.

### EXAMPLE 3

### Behavioral Analysis

Sixteen (16) of nineteen (19) subjects completed the protocol. Over half remained on the program after completion of the pilot study. One subject reported symptomatic improvement of Shy-Drager Syndrome. Generally, subjects reported improvements in a variety of subjective impressions, including:
1. Improved facial expressiveness
2. Improved sleep
3. Improved mobility
4. Improved voice quality and audibility

In summary, subjects in this pilot study of a cyclic exercise protocol experienced some quantitative and qualitative improvements. The cyclic exercise protocol was effectively adapted to a range of subject abilities, from full independence to wheel-chair dependent patients.

### EXAMPLE 4

### Biochemical Analysis

A. Opiate Isolation and Extraction

A 2 ml sample of plasma was obtained from each patient. To each sample was added 20 μl of 10 N HCl and the samples were vigorously vortexed and extracted with 5 ml of 9:1 chloroform:isopropanol. After 5 minutes, the homogenates were certrifuged at 3000 rpm for 15 minutes at room T. The supernatant was collected and dried with a Centrivap Console (Labconco, Kansas City, Mo.). The dried extract was then dissolved in 0.05% trifluoroacetic acid (TFA) water before solid phase extraction. Samples were loaded on a Sep-Pak Plus C-18 cartridge (Waters, Milford, Mass.) previously activated with 100% acetonitrile and washed with 0.05% TFA-water. Elution was performed with a 10% acetonitrile solution (water/acetonitrile/TFA, 89.5%: 10%: 0.05%, v/v/v). Eluted samples were dried in a Centrivap Console and dissolved in water prior to HPLC analysis.

B. HPLC and Electrochemical Identification of the Opiate as Morphine

HPLC analyses of the samples from Part A above were performed with a Waters 626 pump (Waters, Milford, Mass.) and a C-18 Unijet microbore column (BAS, West Lafayette, Ind.) to identify the opiate isolated and extracted in Part A, above. Morphine was identified in the plasma by reverse phase HPLC using a gradient of acetonitrile following liquid and solid extraction and comparison to an authentic standard, as follows.

A flow splitter (BAS) was used to provide the low volumetric flow-rates required for the microbore column. The split ratio was 1 to 9. Operating the pump at 0.5 ml/min. yielded a microbore column flow-rate of approximately 50 μL/min. The injection volume was 5 μL. Detection was performed with an amperometric detector LC-4C (BAS). The microbore column was coupled directly to the detector cell to minimize the dead volume. The electrochemical detection system used a glassy carbon-working electrode (3 mm) and a 0.02 Hz filter (500 mV; range 10 nA). The cell volume was reduced using a 16 μm gasket. The chromatographic system was controlled by Waters Millennium Chromatography Manager V3.2 software and the chromatograms were integrated with Waters Chromatograph Software. The opiate extracted was identified as morphine. Detection sensitivity was 80 picograms.

The amount of morphine in the tissues of the patients was quantified using the method described in Zhu and Stefano in the following manner. The mobile phases were: Buffer A: 10 mM NaCl, 0.5 mM EDTA, 100 mM NaC₂H₃O₂, 50% acetonitrile, pH 5.0. The injection volume was 5 μl. The running conditions were 100% buffer A for the first 10 min., 5% buffer B at 10 min., 50% buffer B at 25 min., and 100% buffer B at 30 min. Both buffers A and B were filtered through a Waters 0.22 μm filter. System temperature was maintained at 25° C. Concentrations were extrapolated from the peak-area calculated for the external standard. The average concentration of morphine in the five samples was 1.43+/−0.58 ng/mL. This result compared favorably with the RIA data. Several HPLC purifications were performed between each sample to prevent residual contamination remaining on the column. Blank runs between morphine HPLC determinations did not show a morphine residue. Furthermore, all fractions corresponding to morphine blank runs were analyzed by Q-TOF mass spectrometry and were found negative.

The morphine extracted from patient plasma samples had the identical retention time when compared to an authentic morphine external standard. This finding was repeated in the samples obtained from five patients from Group 2, none of which had previously been exposed to exogenous morphine.

C. Mass Spectrometry Determination of Morphine

Identification and further characterization of the endogenous opiate alkaloid-like material as morphine was confirmed by nano electro-spray ionization double quadrupole orthogonal acceleration time of flight mass spectrometry (Q-TOF-MS). Mass spectrometry on the samples from Part B above was performed using a Micromass Q-TOF system as follows. One μl of acetonitrile/water/formic acid (50:49:1, v/v/v) containing each sample was loaded in a gold coated capillary using a Micromass F-type needle. The sample was sprayed at a flow rate of 30 nl/min., giving extended analysis time during which a MS spectrum and several MS/MS spectra were run. During this time, fragmentations are generated from a selected precursor ion by collision-induced dissociation (CID). Since not all ions fragment with the same efficiency, the collision energy is typically varied between 20 and 35 V so that the parent ion is fragmented into a satisfying number of different daughter ions. Needle voltage was set at 950 V, and cone voltage at 25 V. The instrument was operated in the positive mode.

The molecular mass attributed to single charged morphine was determined by the analysis to be 286.2 Da, which is consistent with the authentic standard and the theoretical value of 286.14 Da. Fragmentation of both plasma morphine and the authentic standard by CID yielded the same fragments, further confirming that the substance isolated by the extraction set forth in Part A above was morphine.

D. Immunocytochemical Detection of Signaling Molecules

Blood samples were collected from each individual and duplicate plasma samples were stored in EDTA or heparin at −70° C. ACTH concentrations were determined in EDTA plasma. Cortisol and IL-1β analysis required the use of heparinized plasma. For the analysis of IL-10, TNF-α, and IL-6, the type of preservative used on the sample did not matter. Cytokines were analyzed using Pierce/Endogen enzyme-linked immunosorbent assay (ELISA) kits (Pierce/Endogen, Woburn, Mass.). Samples were assayed in duplicate using 50 μl of the appropriate plasma. ACTH and cortisol were analyzed by RIA (ICN Biomedicals, Costa Mesa, Calif.). For the ACH assay, 100 μl of plasma was analyzed in duplicate. For the Cortisol assay, two-25 μl samples were analyzed. Morphine in the plasma was detected with an RIA kit (Diagnostic Products Co., CA). This method was also used to verify the results from the HPLC analysis described above. Four sample groups were analyzed and the data were presented as a mean of a group +/− the standard error. Group 1 contained 19 subjects, Group 2 contained 18, Group 3 contained 17, and Group 4 contained 16. The detection limits for each assay were:

| | |
|---|---|
| Analyte | Limit |

| | | |
|---|---|---|
| Cortisol | ˜1 | μg/dL |
| ACTH | <2 | pg/mL |
| TNF-α | <1 | pg/mL |
| IL-6 | <1 | pg/mL |
| IL-10 | <1 | pg/mL |
| IL-1β | <3 | pg/mL |
| Morphine | 1 | ng/mL |

ACTH plasma levels determined by RIA for Groups 1, 2, 3, and 4 were 76+/−5, 81+/−5, 133+/−23, and 139+/−17 pg/mL, respectively. Groups 3 and 4 exhibited statistically significant increases as compared to Group 1 by the Mann-Whitney rank sum test (p=0.02 for Group 3, p=0.002 for Group 4).

Cortisol plasma concentrations determined by RIA for Groups 1, 2, 3, and 4 were 23+/−2, 18+/−2, 15+/−2, and 22+/−2 μg/dL, respectively. Thus, plasma cortisol levels were not significantly different at any of the observations periods.

Concentrations of the anti-inflammatory cytokine IL-10 were also statistically different for Group 3 as compared to Group 1 (p<0.001). IL-10 concentrations were 1.6+/−0.9, 3.6+/−2.6, 51+/−7, and 74+/−9 pg/mL for Groups 1, 2, 3, and 4, respectively, and exhibited a statistically significant increase in the last two observation periods.

IL-6 concentrations determined for Groups 1, 2, 3, and 4 were 7.5+/−3.8, 4.1+/−2.0, 4.1+/−4.1, and 5.8+/−3.4 pg/mL, respectively. Thus, IL-6 plasma levels remained the same throughout the study period.

IL-1β and TNF-α were not detected in any of the samples analyzed (data not shown).

Plasma morphine concentrations detected by RIA for Groups 1, 2, 3, and 4 were 0.82+/−0.56, 1.48+/−0.67, 1.73+/−0.93, and 1.32+/−0.65 ng/mL, respectively. The differences between Groups 2 (p=0.003), 3 (p=0.001), and 4 (p=0.02) versus Group 1 were statistically significant by two tailed t-test. This result suggests that morphine levels in the plasma were affected by and varied significantly after the start of the exercise protocol.

### Discussion

The foregoing analyses demonstrate that anti-inflammatory molecules significantly increase in the plasma of Parkinson's patients' months after initiating and sustaining a mild exercise protocol. The level of ACTH and IL-10 in the blood increased significantly after one month of exercise. Endogenous plasma morphine levels also significantly increased. IL-6 plasma levels were unchanged and IL-1 and TNF-α levels were unable to be detected during the entire examination period.

Pro-inflammatory cytokines are part of the inflammatory response in the injured brain. Increased expression of pro-inflammatory cytokines such as IL-1, IL-6 and TNF-α has been found in cerebrospinal fluid and in brains of patients with PD. In the mouse model of MPTP-induced PD, increased levels of mRNA for IL-1β, TNF-α, IL-6, IL-10 and INFγ in striatum have been reported. In addition polymorphism of TNF-α genes correlates with early onset of sporadic PD in Japanese patients.

With regard to immune function, patients with PD exhibit changes in their cellular and humoral immune responses. Total lymphocyte count is diminished and there are phenotypic alterations in circulating peripheral blood lymphocytes. The number of CD3+T cells and CD19+B cells is decreased, especially the CD4+subset. The number of memory helper T cells is also decreased, but to a lesser extent, and the percentage of activated helper T cells is increased. Lymphocytes have reduced proliferative response to mitogens such as phytohemaglutinin and concavalin A, demonstrating that cellular immunity is compromised.

Alternation in immune function and cytokine levels are important in PD since pro-inflammatory cytokines can influence catecholamine signaling in the CNS. IL-2 increases ³H-dopamine release in vitro from straital rat slices. In rat hypothalamus in vivo, microglial-derived IL-1 was found to stimulate the release of dopamine and dihydroxyphenyl acetic acid. IL-1β stimulates release of both dopamine and norepinephrine from hypothalami of male rats in vitro. IL-1 in the rat hypothalamus decreases the levels of epinephrine and norepinephrine and elevates their major metabolite, 3-methoxy 4-hydroxyphenylglycol. Also, levels of the dopamine metabolite homovanillic acid are elevated in the rat striatum, hypothalamus and medulla following cytokine administration. Brown and colleagues have also noted a stimulatory effect on norepinephrine metabolism by IL-1β in the rat CNS.

In mice, IL-1 activates the hypothalamic-pituitary-adrenal axis as well as the cerebral catecholamine metaboloism. IL-1 increases the turnover of dopamine in the hypothalamus of lipopolysaccaride (LPS) treated mice. Thus, IL-1-induced activation of the neuroendocrine stress axis persists in LPS-tolerant mice. Cunha demonstrated that IL-8, a molecule also released from activated macrophages, can evoke hyperalgesia in rats by a prostaglandin-independent mechanism. Hyperalgesia can also be evoked in rats by IL-1β.

Glial-derived IL-1 can stimulate the release of corticotropin releasing hormone (CRH) from the paraventricular nucleus of the hypothalamus and release of norepinephrine and dopamine from the hypothalamus. In this scenario, CRH can potentially stimulate proopiomelanocortin (POMC) release from arcuate nucleus neurons, norepinephrine from the locus ceruleus sympathetic nervous system and glucocorticoid production. The increase in ACTH plasma levels delineated here support this scenario as well. Stypula and colleagues also found an increase in plasma ACTH levels in Parkinson's patients, suggesting ACTH's direct or indirect involvement.

Matsubara and colleagues have found an increased level of morphine in the urine of Parkinson's patients after L-dopa administration. The level of morphine in the plasma of Parkinson's patients also increased significantly one month after the start of the mild exercise protocol undergone by the patients in our study. Our previous research has demonstrated the presence of endogenous morphine in various human tissues. Morphine levels increased significantly after surgery in order to down regulate the immune response after trauma, indicating that morphine may play a role in the down regulation of neurological stress.

Monitoring the levels of endogenous morphine in the blood may be accomplished by assaying biological responses induced by the mu3 opiate receptor. Nucleic acid sequences encoding mu3 opiate receptors, polypeptides constituting mu3 opiate receptors, assays employing such receptors and assays identifying agonists and antagonists of such receptors described in detail in PCT Patent Publication WO 99/24471, published 20 May 1999, incorporated by reference here. Biological responses induced by the mu3 opiate receptor include, without limitation, changes in intracellular calcium concentration and nitric oxide release. Intracellular calcium concentrations can be monitored using any method. For example, monitoring can be by using a dye that detects calcium ions. In this case, cells can be loaded with fura-2, a flurorscent dye, and monitored by dual emmission microfluorimetry. Nitric oxide (NO) release can be monitored directly or indirectly using any method known in the art. For example, a NO-specific amperometric probe can be employed to measure directly the NO released from cultured cells or tissue fragments as described elsewhere.

While this invention has been described in conjunction with the specific embodiments outlined above, it is evident that many alternatives, modifications and variations will be apparent to those skilled in the art. Accordingly, the embodiments of the invention as set forth above are intended to be illustrative, not limiting. Various changes may be made without departing from the spirit and scope of the invention as defined in the following claims.

1. A method for assessing the effect of exercise on an individual comprising:
measuring a concentration of at least one compound in the individual's blood before exercise;
measuring a concentration of the at least one compound in the individual's blood after exercise; and
comparing the concentrations of the at least one compound.

2. The method of claim 1, wherein the at least one compound is an anti-inflammatory signal molecule.

3. The method of claim 1, wherein the at least one compound is selected from a group consisting of adrenocorticotropin (ACTH), interleukin-10 (IL-10), and morphine.

4. The method of claim 3, wherein measuring a concentration of morphine comprises assaying a biological response induced by the μ3 opiate receptor.

5. The method of claim 4, wherein the biological response comprises at least one of a change in intracellular calcium concentration and nitric oxide release.

6. A method for treating at least one of a disease and a condition in an individual comprising:
measuring a concentration of at least one compound in the individual's blood before exercise;
directing the individual in an exercise;
measuring a concentration of the at least one compound in the individual's blood after exercise;
comparing the concentrations of the at least one compound; and
modifying the exercise to increase a concentration of the at least one compound.

7. The method of claim 6, wherein the at least one compound is an anti-inflammatory signal molecule.

8. The method of claim 6, wherein the at least one compound is selected from a group consisting of adrenocorticotropin (ACTH), interleukin-10 (L-10), and morphine.

9. The method of claim 8, wherein measuring a concentration of morphine comprises assaying a biological response induced by the μ3 opiate receptor.

10. The method of claim 9, wherein the biological response comprises at least one of a change in intracellular calcium concentration and nitric oxide release.

11. The method of claim 6, wherein the exercise is at least one selected from a group consisting of riding an exercise bicycle, walking on stairs, and bouncing on a trampoline.

12. The method of claim 6, wherein the at least one of a disease and a condition is characterized by at least one of increased blood lymphocytes, increased cerebrospinal fluid lymphocytes, increased immunoglobulin synthesis, increased cytokine levels, increased acute phase protein production, inflammatory processes, sepsis conditions, and viral infection.

13. The method of claim 6, wherein the disease is at least one of Parkinson's Disease, cardiovascular disease, arthritis, pericarditis, vasculitis, lupus, bronchitis, phrenitis, HIV infections, schizophrenia, Alzheimer's Disease, Chrone's Disease, cholitis, diabetes mellitus, autoimmune diseases, toxoplasmosis, asthma, and multiple sclerosis.

14. A method of increasing a concentration of at least one compound in an individual's blood including an exercise regimen comprising:
measuring the individual's resting heart rate;
directing the individual in a first aerobic exercise until the individual's heart rate stabilizes;
directing the individual to rest until the resting heart rate is achieved;
directing the individual in a second aerobic exercise until the individual's heart rate is about 10 beats per minute greater than the stabilized heart rate achieved during the first aerobic exercise;
directing the individual to rest until the resting heart rate is achieved;
directing the individual in a third aerobic exercise such that the heart rate achieved in the second aerobic exercise is achieved in less time;
directing the individual to rest until the resting heart rate is achieved;
directing the individual in a fourth aerobic exercise until the individual's heart rate is about 10 beats per minute greater than the heart rate achieved during the second aerobic exercise;
directing the individual to rest until the resting heart rate is achieved; and
directing the individual in a fifth aerobic exercise until either the individual's heart rate has plateaued or the exercise has continued for one minute.

15. The method of claim 14, wherein the at least one compound is an anti-inflammatory signal molecule.

16. The method of claim 14, wherein the at least one compound is selected from a group consisting of adrenocorticotropin (ACTH), interleukin-10 (IL-10), and morphine.

17. The method of claim 16, wherein measuring a concentration of morphine comprises assaying a biological response induced by the μ3 opiate receptor.

18. The method of claim 17, wherein the biological response comprises at least one of a change in intracellular calcium concentration and nitric oxide release.

19. The method of claim 14, wherein the aerobic exercise is at least one selected from a group consisting of riding an exercise bicycle, walking on stairs, and bouncing on a trampoline.

20. The method of claim 14, wherein the fifth aerobic exercise is about a 9 on the 10-point Borg Scale.

21. The method of claim 14, wherein the exercise regimen is repeated at least about three times per week.

22. The method of claim 21, wherein the individual's first exercise regimen is a baseline regimen and any subsequent exercise regimens are training regimens.

23. The method of claim 21, wherein the method lasts about 12 weeks.

24. The method of claim 22, wherein during the training regimen, the individual's heart rate during the first aerobic exercise is about 70% of the individual's heart rate during the fifth aerobic exercise of the baseline regimen.

25. The method of claim 22, wherein during the training regimen, the individual's heart rate during the fifth aerobic exercise is about 85% of the individual's heart rate during the fifth aerobic exercise of the baseline regimen.

26. The method of claim 22, wherein during the training regimen, the individual's heart rates during the first and fifth aerobic exercises increase during each session of the training regimen.

27. The method of claim 22, wherein during each session of the training regimen, the individual's heart rate during the first aerobic exercise increases about 3 beats per minute and the individual's heart rate during the fifth aerobic exercise increases about 2 beats per minute.

28. The method of claim 22, wherein during the training regimen, the individual's heart rate during the fifth aerobic exercise is about 90% of the individual's heart rate during the fifth aerobic exercise of the baseline regimen.

29. The method of claim 22, wherein during the training regimen, the individual's heart rate during the fifth aerobic exercise is about 95% of the individual's heart rate during the fifth aerobic exercise of the baseline regimen.

30. The method of claim 14, wherein the individual has at least one of a disease and a condition characterized by at least one of increased blood lymphocytes, increased cerebrospinal fluid lymphocytes, increased immunoglobulin synthesis, increased cytokine levels, increased acute phase protein production, inflammatory processes, sepsis conditions, and viral infection.

31. The method of claim 30, wherein the disease is at least one of Parkinson's Disease, cardiovascular disease, arthritis, pericarditis, vasculitis, lupus, bronchitis, phrenitis, HIV infections, schizophrenia, Alzheimer's Disease, Chrone's Disease, cholitis, diabetes mellitus, autoimmune diseases, toxoplasmosis, asthma, and multiple sclerosis.
